(19) United States
(12) Patent Application Publication
(10) Pub. No.: US 2026/0355780 A1
(43) Pub. Date: Nov. 12, 2026

(54) METHOD FOR COVALENTLY BINDING BIOMOLECULES TO PLASTIC

(71) Applicant: CULTURON PTY LTD, Newtown, New South Wales (AU)

(72) Inventors: Marcela BILEK, Sutherland, New South Wales (AU); Kanako COFFI DIT GLEIZE, Killara, New South Wales (AU); Clara Thao Hoang TRAN, Revesby, New South Wales (AU); Shelley Frances Jasmine WICKHAM, Newtown, New South Wales (AU)

(73) Assignee: CULTURON PTY LTD, Newtown, New South Wales (AU)

(21) Appl. No.: 19/099,848

(22) PCT Filed: Aug. 9, 2023
(86) PCT No.: PCT/AU2023/050743

§ 371 Date: Jan. 30, 2025

(30) Foreign Application Priority Data
Aug. 9, 2022 (AU) 2022902232

(51) Int. Cl. B05D 1/00; B05D 3/14; C12N 15/10

(52) U.S. Cl. CPC B05D 1/62; B05D 3/14; C12N 15/1006; B05D 2201/00; C12N 2533/30; C12N 2537/00

(57) ABSTRACT

The present invention relates to methods for covalently binding biomolecules to plastic. In particular, the present invention relates to plasma-activated coatings on plastic wells or dishes to which biomolecules covalently bind.

### FIELD OF THE INVENTION

The present application claims priority from Australian Provisional Patent Application No. 2022902232 (filed 9 Aug. 2022), the contents of which are incorporated in their entirety herein.

The present invention relates to methods for covalently binding biomolecules to plastic. In particular, the present invention relates to plasma-activated coatings of plastic wells or dishes to which biomolecules covalently bind. However, it will be appreciated that the invention is not limited to this particular field of use.

### BACKGROUND OF THE INVENTION

Any discussion of the prior art throughout the specification should in no way be considered as an admission that such prior art is widely known or forms part of common general knowledge in the field.

Microplates provide a fast and convenient platform to study biological processes in high throughput assays, through immobilisation of biomolecules or cells on a solid support. Common applications include enzyme-linked immunosorbent assays (ELISA) and biomolecule interaction studies. The standardised microplate format facilitates robust characterisation via fluorescence and colorimetric detection in automated optical microplate readers with small sample volumes. Commercially available microplates are typically made from plastic (such as polystyrene) that is modified by a tissue culture surface treatment which reduces hydrophobicity and enhances cell adhesion. However, because of the inert nature of polystyrene, additional surface functionalisation is required for immobilisation of biomolecules for solid phase assays. Biomolecules of interest include proteins, such as antibodies and streptavidin, and smaller molecules, such as DNA and peptides. For example, DNA probes are immobilised on microplates for use in accurate DNA detection and as scaffolds for DNA-directed assembly of multienzyme complexes. However, there are limitations of current biomolecule immobilisation methods, such as physical adsorption and chemical activation.

Most commonly, physical adsorption is used to attach biomolecules such as DNA and streptavidin to microplate surfaces through hydrophobic or electrostatic interactions. However, physically adsorbed biomolecules bind weakly to microplates with random orientation and are sensitive to changes in ionic concentration, pH and heat, resulting in reduced reproducibility. Proteins can also be denatured by physical adsorption, for example reducing monoclonal antibody activity to less than 10%.

Stronger and more specific binding can be achieved through chemical activation of microplates, which increases stability, activity, and resistance to washing, and reduces competitive protein exchange. Silane chemistry, for example aminopropyltriethoxylsilane (APTES), is often used for activation of surfaces for biomolecule binding. This surface activation approach has been used in DNA microarrays where the DNA is functionalised with thiol or amine groups prior to chemical coupling to the surface. However, chemical activation methods are multi-step processes, often requiring modified biomolecules such as amine-modified or thiol-modified DNA oligonucleotides and may involve cytotoxic reagents that must be completely removed before downstream applications in cell culture.

Plasma immersion ion implantation (PIII) has previously been used to immobilise of biomolecules on plastic (Bilek & McKenzie, Biophysical Reviews 2010, 2, 55-65; Nosworthy et al., Acta Biomaterialia 2007, 3, 695-704; Kosobrodova et al., ACS Applied Materials & Interfaces 2018, 10, 227-237). In PIII, energetic ions from non-carbon containing gases, such as nitrogen, argon or helium, bombard polymer surfaces to produce a reservoir of radicals in the sub-surface of the polymer to which biomolecules may covalently bind. While this method is fast, reproducible and the reactivity of these surfaces is retained for long periods, it is not suitable for treating the wells of microplates due to shadowing effects caused by the walls of the wells, resulting in uneven reactivity of the plastic surface.

Alternative plasma-based methods, such as argon plasma activation and nitrogen plasma activation have been previously used for microplates (North et al., ACS Applied Materials & Interfaces 2010, 2, 2884-2891; Boulares-Pender et al., Journal of Applied Polymer Science 2009, 112, 2701-2709). However, these methods require chemical activation steps and specific functional groups on the biomolecules. Additionally, the density of active groups incorporated by these approaches is limited by the types of gas mixtures required to produce a mechanically robust surface coating. Another approach involves the direct deposition of nebulized protein, such as collagen, on microplates by dielectric barrier discharge plasma (O'Sullivan et al., ACS Omega 2020, 5, 25069-25076). However, this method requires large quantities of protein and not all biomolecules incorporated into the coating are accessible on the surface.

Consequently, there is a need for fast and reproducible method that covalently binds biomolecules to microplates for long periods.

It is an object of the present invention to overcome or ameliorate at least one of the disadvantages of the prior art, or to provide a useful alternative.

### SUMMARY OF THE INVENTION

It has been surprisingly found that plasma-enhanced chemical vapour deposition (PECVD) using a mixture of carbon-containing gases under a pulsed bias generates a plasma-activated coating (PAC) on a plastic surface that enables covalent immobilisation of biomolecules. The method is fast and reproducible, and the reactivity of the plastic surface is retained for long periods. Additionally, the coverage of reactivity is significantly improved compared to other plasma activation methods (such as PIII) making it suitable for use in Petri dishes and the wells of microplates.

In one aspect, the present invention provides a method for coating a plastic surface, the method comprising:
(a) treating the plastic surface with ions under a pulsed bias; and
(b) depositing plasma on the plastic surface by plasma enhanced chemical vapour deposition (PECVD) of a gas mixture comprising acetylene and argon under a pulsed bias,
wherein a mesh is positioned above the plastic surface such that the plasma passes through the mesh before being deposited on the plastic surface, and wherein the method produces a plasma-activated coating (PAC) on the plastic surface that covalently binds a biomolecule.

In one embodiment, the ions are from noble gases.

In one embodiment the ions are argon ions.

In one embodiment, the gas mixture further comprises nitrogen.

In one embodiment, the atmospheric percentage of nitrogen in the gas mixture is less than 35%.

In one embodiment, the atmospheric percentage of nitrogen in the gas mixture is about 21%.

In one embodiment, the treatment with ions is conducted for about 0.5 to about 30 minutes at RF power of about 5 to about 300 W and negative pulsed bias voltage of about 1 to about 1000 V.

In one embodiment, the treatment with ions is conducted for about 1 to about 15 minutes at RF power of about 50 to about 100 W and negative pulsed bias voltage of about 300 to about 700 V

In one embodiment, the treatment with ions is conducted for about 2 minutes at RF power of about 75 W and negative pulsed bias voltage of about 500 V.

In one embodiment, the plasma deposition is conducted for about 10 to about 30 minutes with a plasma discharge of about 5 to about 300 W and a negative pulsed bias voltage of about 1 to about 1000 V.

In one embodiment, the plasma deposition is conducted for about 10 to about 30 minutes with a plasma discharge of about 10 to about 100 W and a negative pulsed bias voltage of about 300 to about 700 V.

In one embodiment, the plasma deposition is conducted for about 10 to about 30 minutes with a plasma discharge of about 50 W and a negative pulsed bias voltage of about 500 V.

In one embodiment, the gas mixture is maintained at about 110 mTorr.

In one embodiment, the negative pulsed bias is applied with a frequency of about 0.5 to about 10 kHz and pulse duration of about 1 to about 100 μs.

In one embodiment, the negative pulsed bias is applied with a frequency of about 1 to about 5 kHz and pulse duration of about 5 to about 30 μs.

In one embodiment, the negative pulsed bias is applied with a frequency of about 3 kHz and pulse duration of about 20 μs.

In one embodiment, the mesh is formed from a conductive metal.

In one embodiment, wherein the mesh forms the lid of a Faraday cage enclosing the plastic surface.

In one embodiment, the mesh has 50 openings per linear inch.

In one embodiment, the conductive metal is stainless steel.

In one embodiment, the plastic surface has non-flat geometry.

In one embodiment the plastic surface is a microplate well or a Petri dish.

In one embodiment, the plastic is polystyrene.

In one embodiment, the biomolecule is a nucleic acid.

In one embodiment, the biomolecule is a protein.

In one embodiment, the protein is streptavidin or laminin.

In one embodiment, the PAC has a surface nitrogen concentration of about 4 to about 14%.

In one embodiment, the PAC has a relative density of radicals of about 0.016 to about 0.021.

In one embodiment, the PAC has a mean absorption at 450 nm of about 0.052 to about 0.056 a.u.

In one embodiment, the PAC has a thickness of about 20 nm.

In one embodiment, the PAC hybridises DNA at a concentration of about 2×10¹¹ to about 5×10¹¹ molecules/cm².

In one embodiment, the PAC immobilises streptavidin at a concentration of about 2×10¹¹ to about 9×10¹¹ molecules/cm².

In one embodiment, the Faraday cage with a mesh lid comprises an open container, a mesh lid and a cap for securing the mesh lid to the open container, wherein the container, mesh lid and cap are all formed from a conductive metal.

In another aspect, the present invention provides a plastic surface coated with a plasma-activated coating (PAC) when produced by the method of the invention.

In another aspect, the present invention provides a plastic surface coated with a plasma-activated coating (PAC), wherein the PAC has a surface nitrogen concentration of about 4 to about 14%.

In another aspect, the present invention provides a plastic surface coated with a plasma-activated coating (PAC), wherein the PAC has a relative density of radicals of about 0.016 to about 0.021.

In another aspect, the present invention provides a plastic surface coated with a plasma-activated coating (PAC), wherein the PAC has a mean absorption at 450 nm of about 0.052 to about 0.056 a.u.

In another aspect, the present invention provides a plastic surface coated with a plasma-activated coating (PAC), wherein the PAC has a thickness of about 1 nm to about 10 nm.

In another aspect, the present invention provides a plastic surface coated with a plasma-activated coating (PAC), wherein the PAC has a thickness of about 2 nm.

In another aspect, the present invention provides a plastic surface coated with a plasma-activated coating (PAC), wherein the PAC hybridises DNA at a concentration of about 2×10¹¹ to about 5×10¹¹ molecules/cm².

In another aspect, the present invention provides a plastic surface coated with a plasma-activated coating (PAC), wherein the PAC immobilises streptavidin at a concentration of about 2×10¹¹ to about 9×10¹¹ molecules/cm².

In another aspect, the present invention provides a plastic surface coated with a plasma-activated coating (PAC), wherein the PAC binds laminin.

### BRIEF DESCRIPTION OF THE DRAWINGS

FIG. 1: (a) Percentage surface elemental concentration of UT, TC, PIII and PAC treated PS microplates. (b) Relative electron paramagnetic resonance (EPR) integral intensity between UT PS, PIII and PAC treated PS. Measured EPR signal was integrated and normalised by the depth of radical penetration (PIII) or deposition (PAC). (c) Effective depth of the PIII treatment on polystyrene and PAC thickness obtained from ellipsometry measurement of PAC treated silicon wafers. (d) Water contact angles of UT, TC, PIII and PAC treated PS sheets placed in 96 well microplates during plasma treatment. (e) Dispersive and polar components of surface free energy of UT, TC, PIII and PAC treated PS microplates, calculated from the Owens-Wendt-Rabel-Kaelble method. The error bars represent their standard errors. (f) Mean absorbance and autofluorescence with SD of UT (circles), PIII (squares) and PAC (triangles) treated PS microplates. SD bars are hidden when they are smaller than the marker size. The left column and y-axis shows absorbance at 450 nm and the right column and y-axis shows the autofluorescence emission intensity at five excitation/emission wavelengths.

FIG. 2: (a) Schematic diagram showing oligonucleotide immobilisation methods and an example of data plotted on a graph. Fluorescent intensity was measured at steps 1, 3, 5, 7, 8, 9 and 11, and shown on the graph. (b) Distribution map of hybridised DNA on PAC treated microplate by the microplate reader. (c) Baseline subtracted Alexa647 fluorescence intensity of DNA immobilised (pH 4) and hybridised on TC, PIII and PAC treated microplates.

FIG. 3: (a) DNA immobilisation and hybridisation on PIII treated microplates at pH 3-8. (b) DNA immobilisation and hybridisation on PAC treated microplates at pH 3-8. (c) DNA immobilisation and hybridisation to PIII treated microplates 1 week, 1 month and 3 months after treatment. (d) DNA immobilisation and hybridisation to PAC treated microplates 1 week, 1 month and 3 months after treatment.

FIG. 4: DNA immobilisation density on PIII and PAC treated microplates. Cy3 labelled DNA (20×A-GCTCTGCAATCAACTTATCCC-Cy3) was immobilised on plasma treated microplates and Cy3 intensity was measure after washing steps. The trend of immobilised DNA density is similar to hybridised DNA density. This shows that lower hybridisation density on some samples was not caused by the overcrowding of immobilised DNA on the surface.

FIG. 5: Streptavidin immobilisation to microplates. (a) Cy3 fluorescence intensity of streptavidin-Cy3 immobilised on TC, PIII and PAC treated microplates. (b) Ability of immobilised streptavidin to bind with biotin. (c) Streptavidin immobilisation to PIII treated microplates at pH 3-9. (d) Streptavidin immobilisation to PAC treated microplates at pH 3-9. (e) Streptavidin immobilisation to 1 week, 1 month and 3 months old PIII treated microplates. (f) Streptavidin immobilisation to 1 week, 1 month and 3 months old PAC treated microplates.

FIG. 6: Three different conditions, each with 3 mesh sizes, used for plasma treatment to reduce nanoparticle deposition in cell culture plate wells

FIG. 7: The Faraday cage plate holder from top view (A), bottom view (B) and a sketch with dimensions (C).

FIG. 8: Diagram showing plasma treatment chamber containing a plate to be treated with plasma.

FIG. 9: Nanoparticle count using flow cytometry.

FIG. 10: SEM images taken on silicon wafers located inside 24 well plates, plasma treated without a mesh (A, B) and plasma treated with a Faraday cage with 50 mesh lid (C, D). Images were taken at 1000× magnification (A, C) and 5000× magnification (B, D).

FIG. 11: Thickness of PAC measured on silicon wafers located in various wells and plasma deposited using different treatment conditions.

FIG. 12: Comparison of PAC thickness deposited on 24-well plates without a mesh and with a Faraday cage with 50 mesh lid. Shading is used to illustrate the different ranges of thickness.

FIG. 13: Comparison across all three plates showing the percentage surface area coverage of beating colonies observed in each well. 24-well plate with no plasma treatment (Untreated), plasma treated without a mesh (−Mesh) and plasma treated with a Faraday cage with 50 mesh lid (+Mesh). All the plates were incubated with Laminin 521 before cell seeding. Cells were cultured for up to 41 days.

### DEFINITIONS

In describing and claiming the present invention, the following terminology has been used in accordance with the definitions set out below. It is also to be understood that the terminology used herein is for the purpose of describing particular embodiments of the invention only and is not intended to be limiting. Unless defined otherwise, all technical and scientific terms used herein have the same meaning as commonly understood by one having ordinary skill in the art to which the invention pertains.

As used herein the term “about” can mean within 1 or more standard deviation per the practice in the art. Alternatively, “about” can mean a range of up to 20%. When particular values are provided in the specification and claims the meaning of “about” should be assumed to be within an acceptable error range for that particular value.

In the context of the invention the term “subject” includes any human or non-human animal. The term “non-human animal” includes all vertebrates, for example mammals and non-mammals, such as non-human primates, horses, cows, dogs, etc.

In the context of the present invention, the words “comprise”, “comprising” and the like are to be construed in their inclusive, as opposed to their exclusive, sense, that is in the sense of “including, but not limited to”.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

Other than in the operating examples, or where otherwise indicated, all numbers expressing quantities of ingredients or reaction conditions used herein are to be understood as modified in all instances by the term ‘about’.

The recitation of a numerical range using endpoints includes all numbers subsumed within that range (e.g., 1 to 5 includes 1, 1.5, 2, 2.75, 3, 3.80, 4, 5, etc.).

The term “a.u.” means absorbance units.

The term “biomolecule” means a molecule found in living organisms, such as amino acids, lipids, carbohydrates, proteins, polysaccharides and nucleic acids.

The terms “culture plate”, “microplate”, “microtiter plate”, “microwell plate”, “multiwell plate” and ELISA plate means a plate that may contain, e.g., 6, 12, 24, 48, 96, 384 or 1536 sample wells arranged in a 2:3 rectangular matrix. Each well may hold from nanolitres to millilitres of liquid, and can be circular or square.

The terms “Petri dish” and “culture dish” mean a shallow cylindrical dish that may be used to hold growth medium in which cells can be cultured.

The term “Non-flat geometry” refers to structures that are not completely prone or confined on a single plane. Such structures are not substantially flat in shape and possess more than 2 dimensions (e.g., wall(s) and a base). Non-flat geometries include U-shaped, V-shaped and flat bottomed wells, ELISA plates, culture dishes and Petri dishes.

As used herein, the term “pulse duration” means the time over which the pulsed bias is applied in each pulse.

As used herein, the term “Faraday cage” means a container formed from a conductive material.

### PREFERRED EMBODIMENT OF THE INVENTION

Although the invention has been described with reference to certain embodiments detailed herein, other embodiments can achieve the same or similar results. Variations and modifications of the invention will be obvious to those skilled in the art and the invention is intended to cover all such modifications and equivalents.

The present invention is further described by the following non-limiting examples.

### EXAMPLES

### Example 1—Microplate Preparation

In this study, high energy plasma treatment was performed on TC treated polystyrene (PS) microplates (CLS3603) (Sigma-Aldrich) and was compared to unmodified TC treated PS microplates and untreated (UT) PS microplates (CLS3370). All the microplates are made from polystyrene (PS). These microplates have wells with diameter approximately 6 mm and depth approximately 11 mm. There is a 2-3 mm gap between the holder and the bottom of the well when the microplate is placed on either a PIII or a PAC stainless steel holder. Aluminium foil was moulded to fill the gap in order to improve contact to the holder. Microplates were cut into quarters with a hot wire cutter before plasma treatment, so they could be mounted on the sample holder in our small prototype plasma reactor, and were taped back together for DNA and streptavidin immobilisation and fluorescence detection.

### Example 2—PIII Treatment

PIII treatment was performed using inductively coupled radio frequency (RF) power at 13.56 MHz (ENI radio frequency power generator) to generate plasma and a negative voltage bias applied to the stainless-steel sample holder to accelerate positive ions towards the sample. The pressure inside the chamber was evacuated to less than 5×10⁵ Torr and high purity nitrogen gas was introduced and maintained at 2 mTorr during the treatment. The RF forwarded power was 100 W with a reverse power of 12 W when matched. Microplates, with or without aluminium foil, were taped on the stainless-steel holder with stainless-steel mesh placed 5 cm from the holder and electronically connected to it. Nitrogen ions were accelerated through the mesh towards the stainless-steel holder with 20 kV negative bias pulses of 20 μs duration at a frequency of 50 Hz. The microplates were treated for 400 or 800 seconds (PIII 400 or PIII 800).

### Example 3—PAC Treatment

PAC treatment was performed in a separate plasma system using capacitively coupled RF power at 13.56 MHz (Eni OEM-6) and a negative pulsed bias generated by RUP6 pulse generator (GBS Elektronik GmbH, Dresden, Germany). Microplates were positioned on a stainless-steel sample holder connected to RUP6 with aluminium foil placed under the microplates to eliminate air gaps between the sample holder and the plate and strengthen the accelerating electric field at the surface of the plate. The chamber was evacuated to less than 5×10⁻⁵ Torr. Prior to the plasma coating, microplates were activated with argon ions to facilitate coating adhesion for 2 minutes at RF power of 75 W and an applied bias of 500 V. The pressure of argon inside the chamber was maintained around 70-80 mTorr. For PAC deposition a reactive gas mixture of acetylene, nitrogen and argon was introduced into the chamber. The ratios between gases were controlled by a mass flow controller (Allicat Scientific) and the pressure inside the chamber during the coating deposition was maintained at 110 mTorr. Plasma deposition was conducted with a plasma discharge of 50 W and a negative bias voltage of 500 V for 10 or 30 minutes. Negative bias from RUP 6 was applied with a frequency of 3 kHz and pulse duration of 20 μs. Four different gas ratios were chosen for comparison: no nitrogen (0% nitrogen), low nitrogen (21% nitrogen), moderate nitrogen (71% nitrogen), and high nitrogen (93% nitrogen) (Table 1). Separate PAC treatments were performed on smooth silicon wafers with a native oxide layer to measure the coating thickness.

TABLE 1
PAC treatments with four different gas flow rate recipes.

| | Gas flow rate (SCCM) | | | at % |
|---|---|---|---|---|
| Treatment | Acetylene | Nitrogen | Argon | nitrogen |
| No | 1 | 0 | 13 | 0 |
| nitrogen | | | | |
| Low | 1 | 3 | 10 | 21 |
| nitrogen | | | | |
| Mod | 1 | 10 | 3 | 71 |
| nitrogen | | | | |
| High | 1 | 13 | 0 | 93 |
| nitrogen | | | | |

Upon exposure to air, radicals on the treated surfaces react with oxygen in the air to form oxygen containing groups. These changes to the surface chemistry saturate after a week and, therefore, after each treatment the microplates were covered in aluminium foil and stored for at least a week in air at room temperature before all subsequent analysis and biomolecule immobilisation.

### Example 4—Chemical Composition of PAC and PIII Modified Microplates

Ellipsometric spectroscopy was used to calculate the coating thickness of PAC. Silicon wafers were used as substrates to measure coating thickness and coatings were deposited with the various PAC recipes shown in Table 1. Ellipsometric data were collected at three angles of incidence (65°, 70°, and 75°) using a J.A Woollam M2000 V spectroscopic ellipsometer. A model consisting of a silicon substrate, silicon oxide layer (2 nm), and a Cauchy layer to represent the PAC layer was used to fit the data to obtain the film thickness.

The surface free energies of PIII and PAC treated TC treated 96 well microplates (1 week post treatment) were calculated and compared to untreated polystyrene. Contact angle measurements using two liquid probes (water and diiodomethane) were performed using a Theta Tensiometer (Biolin Scientific). Results are averaged over 10 drops for each sample. Surface free energies were calculated using Owens-Wendt-Rabel-Kaelble method.

In order to measure radical densities after PIII and PAC treatment of microplates, polystyrene film (Goodfellow, thickness 0.19 mm) was cut into 40 mm×5 mm strips and treated with either PIII or PAC. Polystyrene film was used as a proxy for the microplate, which did not fit in the measurement instrument. The microwave absorption by unpaired electrons from the samples were measured using an electron paramagnetic resonance (EPR) spectrometer (Bruker EMX X-band). Measurements were done at room temperature with a microwave power of 2 mW and a frequency of 9.8 MHz. A magnetic field was scanned with a central value of 3523 G and a sweep width of 200 G. Ten scans were measured on each sample. Similar measurement was applied to 2,2-diphenyl-1-picrylhydrazyl (DPPH) powder containing EPR tube with known radical density to calculate the number of unpaired electrons on each sample.

Changes in surface chemistry after PIII and PAC treatment of microplates were determined by comparison of FTIR-ATR spectra of un-treated and treated polystyrene. Polystyrene films were cut into small discs and placed at the bottom of microplate wells for PIII and PAC treatment. A week after the treatment, the discs were analysed using a Hyperion FTIR spectrometer (Bruker) equipped with a micro germanium crystal ATR accessory. Each analysis consists of 128 scans with a resolution of 4 cm⁻¹. The spectra were normalised with the intensity of the 1490 cm⁻¹ peak of polystyrene for comparison.

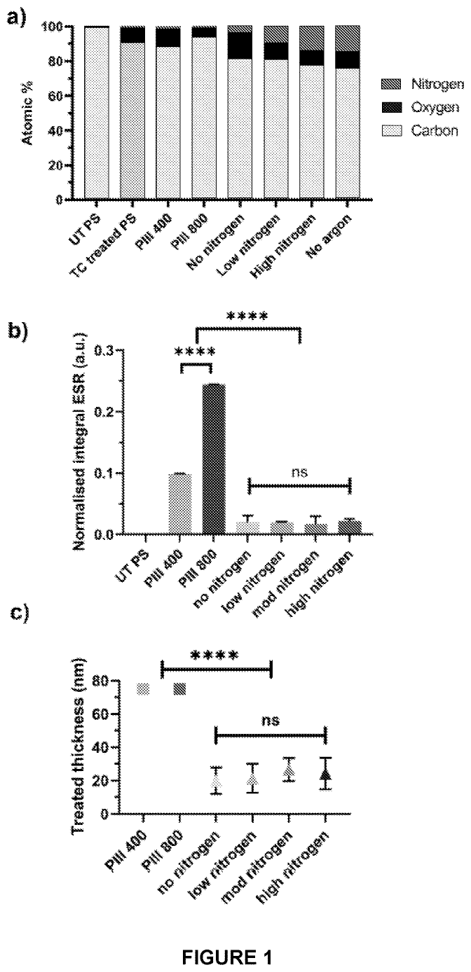

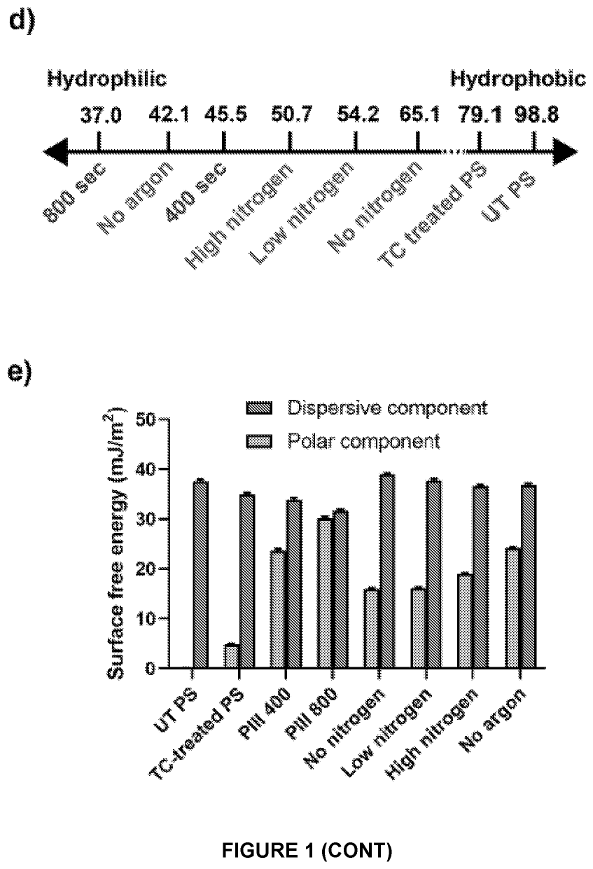

The chemical composition of PIII treated microplates was determined by XPS, comparing 400 s (PIII-400) and 800 s (PIII-800) treatment times (FIG. 1a). The oxygen content of the PIII treated microplates (6.1-11.1%) was not significantly different to the TC-treated microplate (8.3%). Similarly, surface nitrogen content was very low both before (0.6%) and after PIII treatment (0.5-0.8%). This was surprising, as previous studies have shown an increase in nitrogen content (7-12%) following 400 s PIII treatment of glass coverslips spin-coated with polystyrene (Kosobrodova et al., ACS Applied Materials & Interfaces 2018, 10, 227-237) and an increase in oxygen composition from exposure to air following treatment of polystyrene film (Kosobrodova et al., Nuclear Instruments and Methods in Physics Research Section B: Beam Interactions with Materials and Atoms 2013, 304, 57-66.41). Differences in the results observed here may be due to the deep well shape or differences in the type of polystyrene structure, such as the orientation of phenol groups, of the microplates as compared to polystyrene film or spin-coated layers.

Long-lived radicals appearing in the modified surface layer have been proposed as the main mechanism underlying covalent immobilisation of biomolecules on PIII surfaces (Bilek et al., Proceedings of the National Academy of Sciences of the United States of America 2011, 108, 14405-14410). Radical densities of radicals in the samples can be calculated from EPR intensities by comparing with that obtained from a standard DPPH sample with known radical density. However, due to the difference in geometry and volume (DPPH powder was put in an EPR tube while PIII and PAC treated PS strips were mounted on an EPR tube), we could not calculate the exact radical density on our samples. Instead, we compared the relative density of radicals in those samples by normalising with the depth of the plasma treatment with the assumption that the measurement areas are the same for all samples.

For PIII samples, the depth of treatment was assumed as 75 nm from the literature (Gan et al., Nuclear Instruments and Methods in Physics Research Section B: Beam Interactions with Materials and Atoms 2006, 247, 254-260.39). The relative density of radicals on treated microplates was found here to increase significantly (one-way ANOVA, p<0.0001) after both 400 and 800 s PIII (0.10±0.001 and 0.24±0.0003 respectively) treatments compared to UT PS microplates (1.12×10⁻⁶, FIG. 1b). The increase from PIII-400 to PIII-800 was also significant (one-way ANOVA, p<0.0001).

Wettability of surfaces is another property known to be affected by PIII treatment. Here, PIII treatment was observed to significantly (one-way ANOVA, p<0.0001) decrease the water contact angle of PIII-400 (45.5±1.7°) and PIII-800 (37.0±1.9°) treated microplates, compared to TC-treated microplates (79.1±0.6°) (FIG. 1d). The Owens-Wendt-Rabel-Kaelble method considers the surface energy to be comprised of a polar component and a dispersive component. Polar components of PIII treated microplates (23.7±0.3-30.1±0.3 mJ/m²) were found to be significantly higher than TC-treated microplates (4.8±0.1 mJ/m², one-way ANOVA, p<0.0001), and increased with treatment time (FIG. 1e). The wettability and polar surface energy component values for the PIII treated microplates had a stronger correlation to the relative density of radicals as described above, than to the surface nitrogen and oxygen composition.

The chemical composition of PAC microplates was determined by XPS, comparing four different gas recipes containing increasing amounts of nitrogen; no nitrogen, low nitrogen, moderate (mod) nitrogen and high nitrogen (FIG. 1a). Surface nitrogen was found to increase with % of nitrogen gas across all PAC recipes. Values measured were TC (0.6%), to no nitrogen (4.0%), low nitrogen (9.7%), mod nitrogen (13.8%), and high nitrogen PAC (14.6%). For surface oxygen, no nitrogen PAC treated microplates had increased surface oxygen (14.9%). All other recipes had oxygen content (8.5-9.4%) similar to that of TC−treated microplates (8.3%). It is important to note that the sampling depth measured by XPS (about 10 nm) may include the polystyrene substrate below the deposited layer, which would lower the nitrogen percentage.

There was no significant difference between the relative density of radicals in different PAC treated samples (FIG. 1b, no nitrogen 0.020±0.01, low nitrogen 0.019±0.0008, mod nitrogen 0.016±0.013, high nitrogen 0.021±0.0035). As described above, these values were normalised with the PAC coating thickness (FIG. 1c).

The water contact angles of the microplates decreased after PAC treatment (FIG. 1d). There was no significant difference in water contact angles between low and mod nitrogen PAC, but the hydrophilicity of high nitrogen was significantly higher than that of no nitrogen (one-way ANOVA, p<0.0001). The water contact angles ranged between 42.1° and 65.1°. The polar component of PAC samples also increased with increasing amount of nitrogen in the PAC recipe.

Overall, PIII microplates were found to have low surface nitrogen composition, high relative density of radicals and were very hydrophilic. PAC microplates had high nitrogen composition, moderate relative density of radicals and were more hydrophilic than untreated plates. PIII and PAC treated microplates were found to differ in a number of measured parameters. PAC treated microplates had significantly higher amounts of surface nitrogen compared to PIII treated microplates (PIII 0.5-0.8%, PAC 4-14.6%), while PIII treated microplates had higher relative density of radicals (PIII 0.10-0.24, PAC 0.016-0.021) and were more hydrophilic (37.0°-45.5°) compared to all PAC (50.7°-65.1°) except the high nitrogen treatment (42.1°). The relative density of radicals appeared to have a greater correlation with surface hydrophilicity than surface nitrogen and oxygen compositions.

### Example 5—Optical Properties of PIII and PAC Modified Microplates

Changes in optical properties can impact the utility of plasma-activated microplates, because optical readout is the main method of detection for biochemical plate assays. Changes in absorbance and autofluorescence after PIII and PAC treatment of microplates were measured with PHERAstar FSX (BMG Labtech) and compared to untreated microplates. Absorbance was measured with top optic at 450 nm with 22 flashes per well. Fluorescence intensity was measured at five commonly used excitation and emission wavelengths with five optic modules (460/510, 485/520, 540/580, 575/620, 635-20/680-20). For each end point measurement, the top optic was used to scan the centre of the well with 10 flashes, focal height was adjusted for each sample and the gain was set at 1400.

The effects of PIII and PAC treatment on absorbance and autofluorescence were characterised (FIG. 1f). Absorbance was measured at 450 nm as this wavelength is used in many assays such as HRP colorimetric assay. Mean absorbance at 450 nm was found to be significantly higher (one-way ANOVA, p<0.0001) for PIII (0.150-0.207 a.u.) and PAC (0.052-0.056 a.u.) compared to untreated microplates (0.046±0.001 a.u.). The greatest increase in the absorbance was observed for PIII-800 (0.207±0.008 a.u.), followed by PIII-400 (0.150±0.010 a.u.). Browning of the clear microplates by PIII treatment was also visible by eye and increased with treatment time. This browning may be due to clustering of carbonised structures in the modified layer, which increase in number and size with longer treatment times. PIII samples with aluminium had smaller variance in absorbance over multiple wells (0.016 a.u.) compared to the non-aluminium samples (0.030 a.u.), suggesting that the aluminium modification improved the uniformity of treatment across the microplate. PAC treated microplates had much smaller increase in absorbance (1.13-1.22 fold) compared to PIII treated microplates (3.36-4.5 fold), with no significant difference between PAC gas treatments (0.052-0.056 a.u., one-way ANOVA, p>0.1). Overall, PIII resulted in a large (288%) increase in absorbance at 450 nm relative to untreated microplate, while PAC had a more modest (17.5%) relative increase.

Generally, aromatic polymers have high autofluorescence at shorter wavelength due to low energy π to π* transitions. This agrees with observations here, with all untreated and treated microplates having higher autofluorescence at shorter wavelengths (460/510 and 485/520 nm) than at longer wavelengths (540/580, 575/620, 635/680 nm). In addition to this trend, PAC treated microplates also had significantly higher autofluorescence at shorter wavelengths (162-196, one-way ANOVA, p<0.0001) compared to untreated (155-157), while PIII treated had significantly lower (100-144, one-way ANOVA, p<0.0001). In PIII treatment, the aromatic rings are partially amorphised which explains the lower autofluorescence observed for PIII and supported by the observation that PIII-800 had lower autofluorescence (100-118) than PIII-400 (126-144). In contrast, at longer wavelengths, there is less difference in autofluorescence across all samples. Overall PAC and PIII treatments changed microplate autofluorescence at shorter wavelengths (510-520 nm), but they had smaller changes at longer wavelengths (580-680 nm). The changes at shorter wavelengths were small relative to total autofluorescence from the polystyrene substrate, with PAC giving an 8% increase and PIII a 27% decrease. At longer wavelengths, PAC had a 9% increase and PIII had a 14% decrease from untreated polystyrene.

### Example 6—DNA Oligonucleotide Immobilisation for PIII and PAC Modified Microplates

For applications in DNA-binding assays it is important for the immobilised DNA to maintain its ability to hybridise with complementary DNA. Therefore, measurements focused on the density of hybridised DNA, as detected by an Alexa-647 fluorophore covalently attached to the hybridising DNA oligonucleotide.

A twenty-one nucleotide (nt) ssDNA sequence (IDT DNA) was designed with a linker of 20 additional adenine nucleotides (Table 3). It was expected that in acidic conditions the amine groups in the adenine nucleotides of the linker will be protonated, and the linker will therefore be preferentially electrostatically attracted to the negatively charged plasma surface as compared to the core DNA sequence.

Wells of untreated, PIII and PAC treated 96-well microplates were incubated with 40 μL of 2 μM ssDNA in 10 mM of citric acid/sodium citrate buffer at pH 3, 4, 5 or 6 or disodium hydrogen phosphate/sodium dihydrogen phosphate buffer at pH 7 or 8 for one hour at room temperature on a shaker. DNA solution was replaced with 200 μL of 1% BSA in 10 mM PBS at pH 7.4 for one hour at room temperature on a shaker to block the remaining active surface. BSA solution was removed and the wells were washed with 200 μL of 2% SDS three times with vigorous shaking. After rinsing with 200 μL of MilliQ water three times, DNA hybridisation was performed by adding a 21-nt complementary DNA strand with 3′ Alexa647 fluorophore modification (IDT DNA). Complementary DNA was added to a hybridisation buffer (consisting of 2 mM magnesium chloride (Sigma-Aldrich), 1×Tris EDTA (Sigma-Aldrich), 1% BSA and 0.6% SDS) to a final concentration of 0.8 μM. Each well was incubated with 40 μL of 0.8 μM complementary DNA solution for one hour on a shaker at room temperature. After removing the solution, the wells were washed with 200 μL 10 mM PBS three times with vigorous shaking. Then, the wells were washed three times with vigorous shaking with 200 μL of each of 3 washing buffers; washing buffer 1 (2×saline-sodium citrate (SSC)+0.6% SDS), washing buffer 2 (0.2×SSC+0.6% SDS), and washing buffer 3 (0.1×SSC+0.5% Tween 20). After rinsing the wells with 200 μL of 10 mM PBS, each well was filled with 40 μL of 10 mM PBS for fluorescence intensity measurement. The fluorescence intensity of the Alexa647 modification on the complementary DNA was measured with an 635-20/680-20 optic module on PHERAstar FSX. For each measurement, the top optic was used to scan a 10×10 matrix of well diameter 3-5 mm with 10 flashes at each scan point. Focal height was adjusted for each sample and the gain was set at 2000.

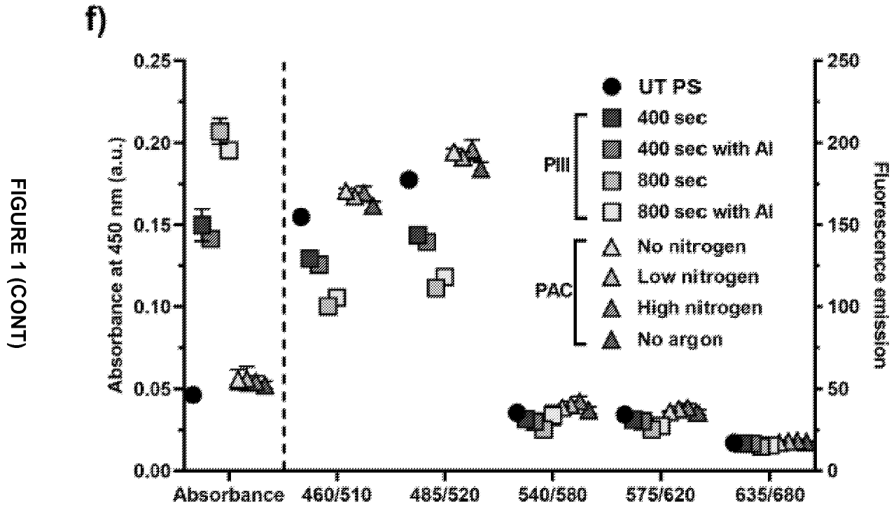

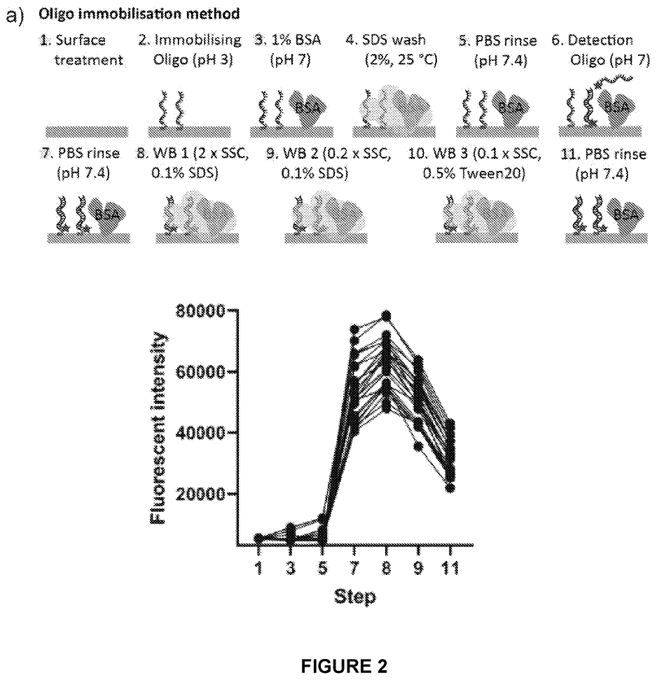

To correctly detect only DNA hybridised to covalently immobilised DNA, wash steps were required to remove non-specifically bound DNA. Wash steps were included to remove non-covalently bound ssDNA (step 4, FIG. 2a) and non-hybridised ssDNA from the microplates (step 7-11, FIG. 2a). Washing was found to be effective at reducing the fluorescence signal of negative controls back to the baseline level (t-test, p<0.0001), for both controls with no immobilising ssDNA (step 2) or addition of the wrong sequence of hybridising DNA (step 6, FIG. 2c). For example, giving a 50% decrease from step 7 to 11, which is equivalent to approximately 8.4×10¹⁰ DNA molecules removed from the surface. In preliminary measurements, the density of hybridised DNA was found to be higher in the centre of the microplate wells compare to the edges (FIG. 2b). Well edges have less efficient fluid mixing and experience lower flux of implanting ions due to shadowing and the reduction of sheath area to wall area ratio, which would limit DNA density near the edge. Therefore, measurements of hybridisation density were averaged over a circle of 3 mm diameter at the centre of the wells, which had a total diameter of 5 mm.

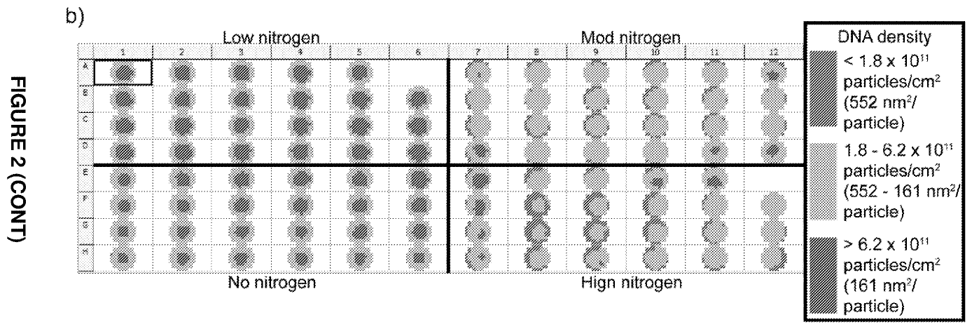

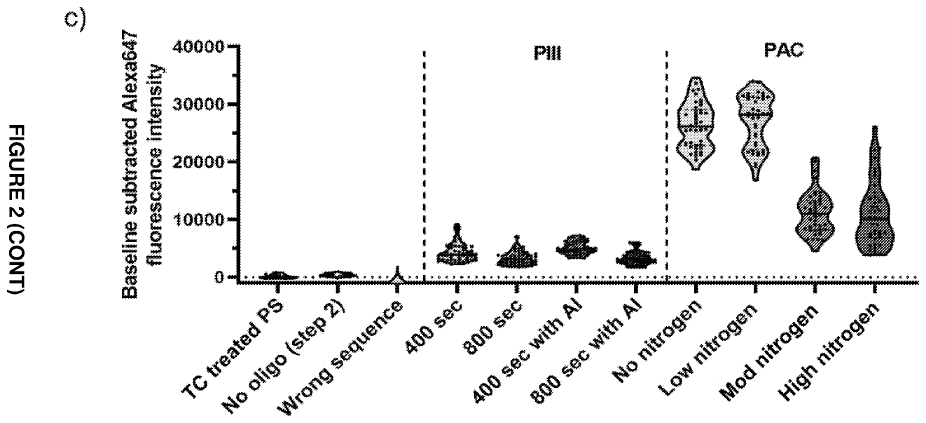

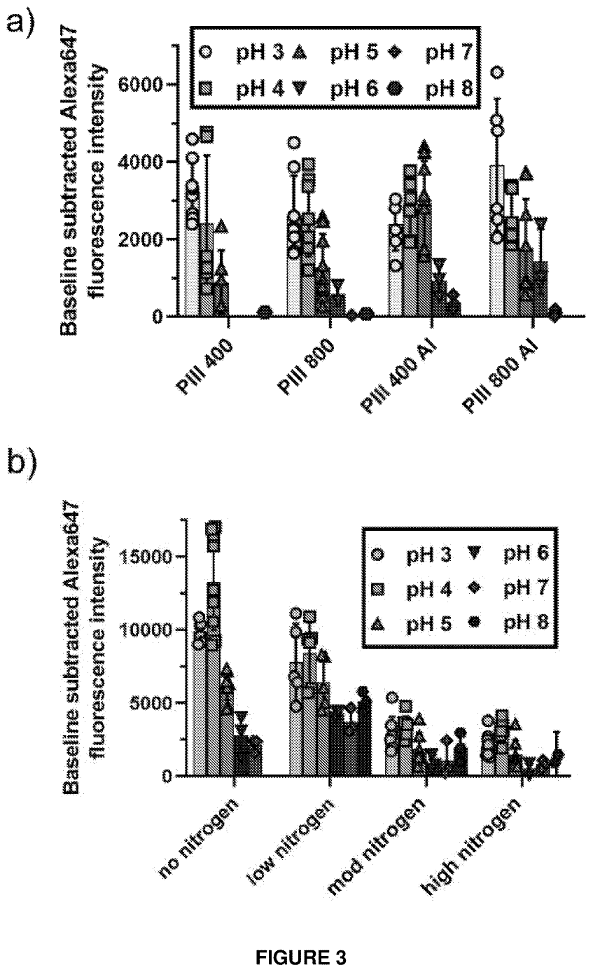

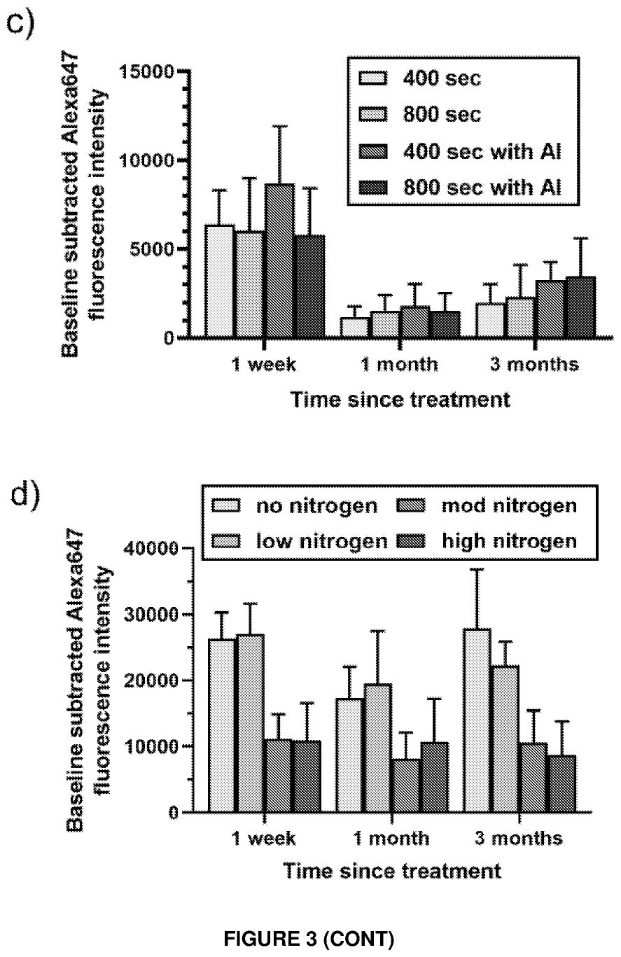

DNA hybridisation was characterised for all recipes of PIII (PIII-400, PIII-800) and PAC (no, low, mod and high nitrogen). Greater fluorescence signal, indicating higher DNA hybridisation density, was observed for all plasma treated samples compared to the negative controls (<3.1×10¹⁰ molecules/cm²), and was also higher on PAC than PIII treated microplates (PIII 1.3×10¹¹ molecules/cm², PAC 3.4×10¹¹ molecules/cm², one-way ANOVA, p<0.0001) (FIG. 2c, Table 2). DNA hybridisation was approximately 2-5 fold denser on PAC compared to PIII (1.1-1.6×10¹¹ molecules/cm², one-way ANOVA, p<0.0001), and no nitrogen and low nitrogen PAC recipes had the greatest amount of hybridised DNA overall (4.7-4.8×10¹¹ molecules/cm², one-way ANOVA, p<0.0001). There was no significant difference between the four different PIII surfaces, indicating that the aluminium foil and the treatment time did not affect DNA immobilisation. The effect of pH on DNA immobilisation was tested by incubating immobilising ssDNA in pH 3 to 8 (step 2, FIG. 2a). The highest hybridisation density was observed for pH 3 and 4 for almost all PIII and PAC treated microplates (FIG. 3a, b). DNA immobilisation capability of PIII treated microplate decreased one month after the PIII treatment, but there was no further reduction up to three months (FIG. 3c). All PAC samples retained the same DNA immobilisation capability up to three months (FIG. 3d). DNA immobilisation and hybridisation on no and low nitrogen PAC samples were further optimised by using freshly prepared hybridisation solution and measuring fluorescence intensity from 3 mm diameter instead of 5 mm diameter. After further optimisation, the average hybridised density on no and low nitrogen increased to 5.2×10¹² molecules/cm² (FIG. 4).

Interestingly, the measured relative density of radicals was found not to correlate with the amount of DNA hybridised to the surface. PIII microplates had 5-15 fold higher relative density of radicals than PAC, but a 2-5 fold lower DNA hybridisation capacity. This demonstrates that the low amount of radicals on PAC surfaces was sufficient for dense DNA immobilisation.

TABLE 2
Hybridised DNA and immobilised streptavidin
densities of TC, PIII and PAC microplates

| | Hybridisation | |
|---|---|---|
| Surface | density (×10¹¹ | SA density (×10¹¹ |
| modification | molecules/cm²) | molecules/cm²) |
| TC treated | 0.3 | 0.01 |
| PIII-400 | 1.2 | 0.64 |
| PIII-800 | 1.1 | 0.61 |
| PIII-400 with Al | 1.6 | 0.84 |
| PIII-800 with Al | 1.1 | 0.79 |
| PAC - no nitrogen | 4.8 | 2.2 |
| PAC - low nitrogen | 4.7 | 9.0 |
| PAC - mod nitrogen | 2.0 | 4.3 |
| PAC - high nitrogen | 2.0 | 4.0 |

PAC surfaces had higher DNA density than PIII, which could be explained by the higher composition of nitrogen that could be protonated on PAC surfaces leading to more favourable electrostatic interactions. The highest DNA density was found on PAC surfaces with lower composition of surface nitrogen.

Hydrophilicity of the surface is another important factor that could affect the intermolecular interaction with DNA. Both hydrophilic and hydrophobic surfaces can attract DNA. Hydrophobic surfaces can attract nearby DNA through short range vdW forces, and hydrophilic surfaces can attract longer-range DNA via dipole-dipole and hydrogen bonding. However, hydrophilic surfaces are more susceptible to form a dense hydration layer which competes with intermolecular attraction between the surface and DNA via hydrogen bonding. Thus, higher DNA density on the no nitrogen and low nitrogen PAC surfaces compared to other samples could be explained by their relative hydrophobicity compared to mod and high nitrogen PAC and all PIII conditions, which minimises formation of the hydration layer and maximises vdW forces.

### Example 7—Streptavidin Immobilisation for PIII and PAC Modified Microplates

Streptavidin immobilised on plasma treated surfaces was directly detected by the fluorescence of covalently modified Cy3 streptavidin. For PAC surfaces it was also indirectly detected by binding with dual biotin-modified Alexa647-modified DNA strands. While the former quantifies protein immobilised, the latter will only detect active protein on the surface.

Wells of Pill-treated, PAC-treated and untreated microplates were incubated with 40 μL of 10 μg/mL streptavidin-Cy3 (Sigma-Aldrich, P6402) in 10 mM of citric acid/sodium citrate buffer at pH 3, 4, 5 or 6 or disodium hydrogen phosphate/sodium dihydrogen phosphate buffer at pH 7 or 8 for one hour at room temperature on a shaker. Streptavidin solution was replaced with 200 μL of 1% BSA in 10 mM PBS at pH 7.4 for one hour at room temperature on a shaker to block the remaining active surface. BSA solution was removed and the wells were rinsed with 200 μL of 10 mM PBS three times. After rinsing, each well was washed with 200 μL of 10% Triton in 10 mM PBS three times. Next the wells were rinsed with 200 μL of 10 mM PBS a further three times, then the wells were incubated with 2 μM 5′ biotin-modified ssDNA (IDT DNA, Table 3) in 10 mM PBS at pH 7.4 for one hour at room temperature on a shaker. Following biotin-DNA incubation, the wells were incubated with the Alexa647-modified complementary DNA, as described in the DNA immobilisation method. The hybridised surface was washed with 200 μL of washing buffer 4 (2×SSC+0.05% Tween 20) three times before rinsing with 10 mM PBS. PHERAstar FSX fluorescence plate reader with optic modules 540/580 and 635-20/680-20 was used to detect immobilised streptavidin-Cy3 and the hybridised complementary Alexa647-DNA, respectively. For each measurement, the top optic was used to scan 10×10 matrix of well diameter 3-5 mm with 10 flashes at each scan point. Focal height was adjusted for each sample and the gain was set at 1000.

TABLE 3
Sequences of immobilising DNA, hybridising DNA and biotin DNA to
bind to immobilised streptavidin

| DNA name | DNA sequence |
|---|---|
| immobilising DNA | 20xA-GCTCTGCAATCAACTTATCCC |
| hybridising DNA | GGGATAAGTTGATTGCAGAGC-Alexa647 |
| wrong hybridising DNA | GTGATGTAGGTGGTAGAGGAA-Cy3 |
| biotin DNA | biotin-GCTCTGCAATCAACTTATCCC |

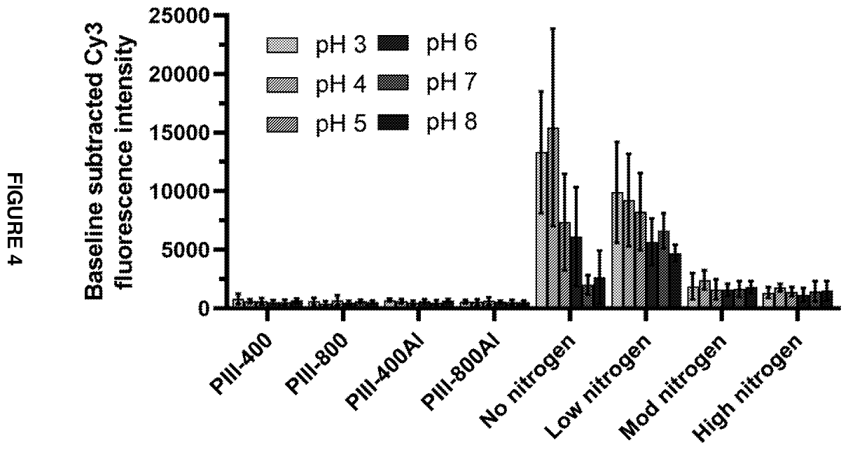

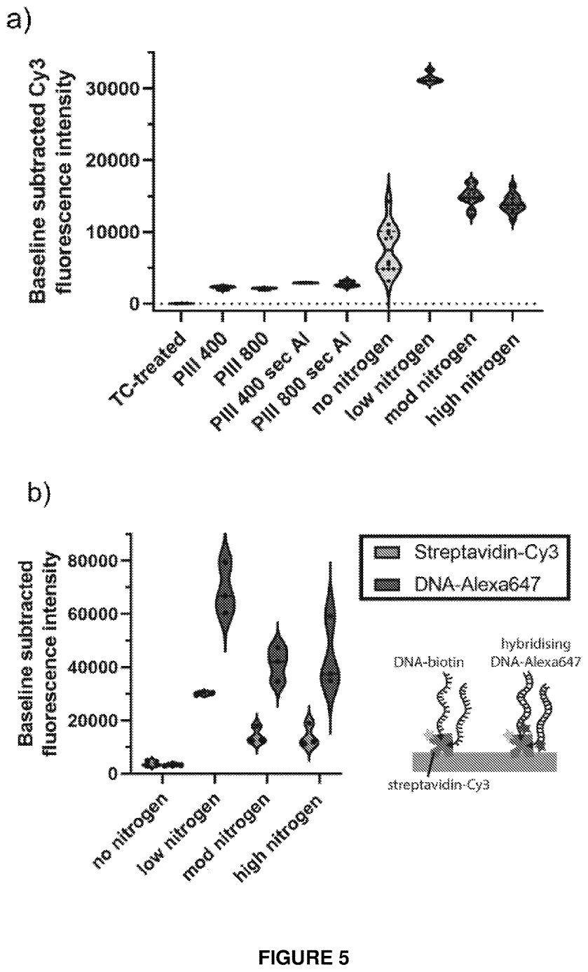

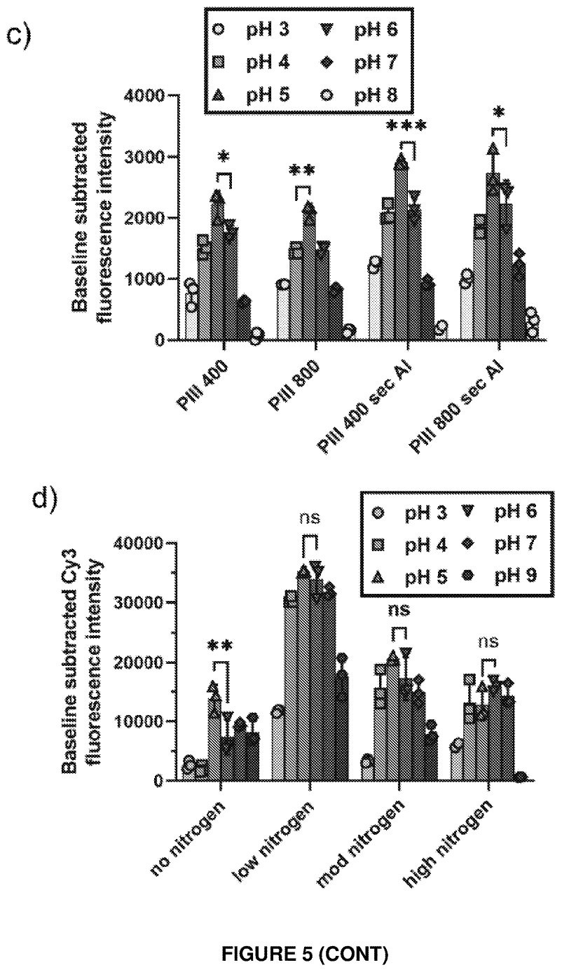

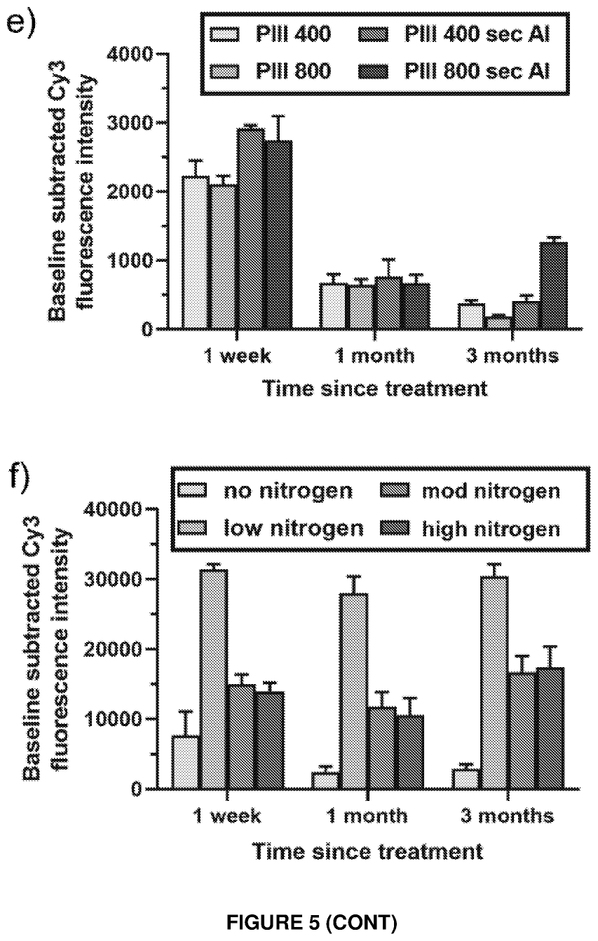

Similarly to DNA, streptavidin was also found to bind more on PAC treated microplates than PIII treated microplates (FIG. 5a), with significant increase for all plasma treated conditions above the non-treated control (TC treated 0.01×10¹¹ molecules/cm², Pill 0.7×10¹¹ molecules/cm², PAC 4.9×10¹¹ molecules/cm², one-way ANOVA, p<0.0001). In contrast to DNA, low nitrogen PAC had the highest amount of bound streptavidin (no nitrogen 2.2×10¹¹ molecules/cm², low nitrogen 9.0×10¹¹ molecules/cm², mod nitrogen 4.3×10¹¹ molecules/cm², high nitrogen 4.0×10¹¹ molecules/cm²), whereas for DNA both low and no nitrogen PAC were similarly high. This PAC trend was reproduced for biotin-DNA binding, indicating that the immobilised protein remains active (FIG. 5b, no nitrogen×10¹¹, low nitrogen 12.2×10¹¹, mod nitrogen 7.3×10¹¹, high nitrogen 7.8×10¹¹ biotin-DNA molecules/cm²). By calculation, there were an average of 2.14 hybridised DNA-biotin molecules bound to each immobilised streptavidin on the surface. The best PIII surface for streptavidin immobilisation was PIII-400 with aluminium, but it had about 10-fold less streptavidin compared to the best PAC condition. The immobilisation pH had an effect on the amount of immobilised streptavidin on PIII and PAC microplates. For PIII microplates, the optimum immobilisation was achieved at pH 5 (FIG. 5c). For PAC microplates, optimum streptavidin immobilisation was achieved at pH 5 for no nitrogen PAC, and at pH 4-7 for the other PAC recipes (FIG. 5d). Streptavidin immobilisation deteriorated significantly after one month for all PIII samples (FIG. 5e). However, the amount of immobilised streptavidin did not change significantly for PAC samples up to 3 months old (FIG. 5f).

### Example 8—Comparison of PIII and PAC Modified Microplates

PAC surfaces were found to have more nitrogen and lower radical density, and were more hydrophobic and more stable over 3 months than PIII surfaces. Optimal conditions were obtained for high density DNA (PAC, 0% or 21% nitrogen, pH 3-4) and streptavidin (PAC, 21% nitrogen, pH 5-7) binding. PAC activated microplates allow for high density covalent immobilisation of functional DNA and protein in a single-step, without specific linker chemistry.

### Example 9—Preventing Accumulation of Plasma-Generated Nanoparticles

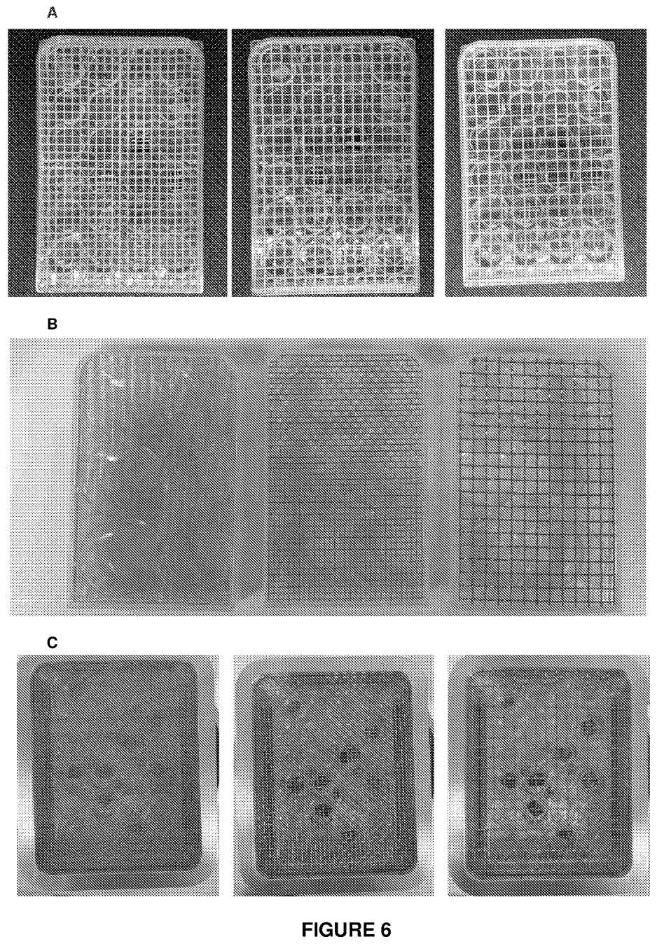

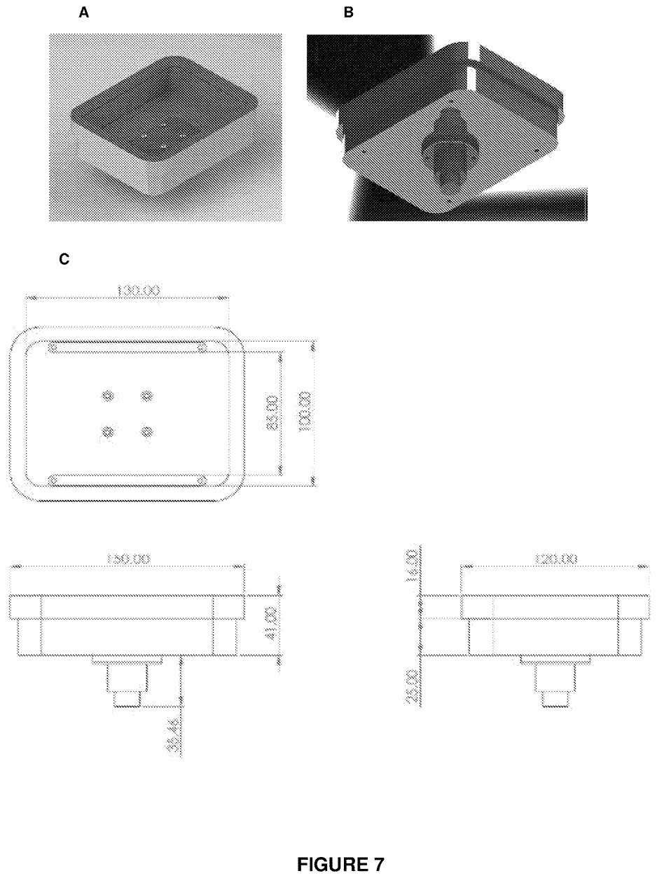

Plasma-generated nanoparticles were observed to accumulate in PAC coated 24 well plates. To prevent plasma-generated nanoparticles falling into the wells, the configuration of the sample holder was modified. Plasma treatments on 24 well plates were conducted using 3 conditions described below and shown in FIG. 6.
Polymer mesh: square mesh with 3 sizes (3, 4 and 5 mm spacing) were modified from the lid of tissue culture plate using laser cutting and fit on the well plate during plasma treatment.
Electrically conductive mesh: stainless steel mesh with 3 sizes, 4 mesh (i.e., 4 openings per linear inch), 10 mesh (i.e., 10 openings per linear inch) and 50 mesh (i.e., 50 openings per linear inch), was cut to cover the plate. The meshes are made of stainless-steel wire woven to create square gaps with the spacing size varying from 6 mm to 0.45 mm. A polymer fitting cut out from the lid of tissue culture plate was used to secure the mesh on top of the plate. The stainless steel mesh was not electrically connected to the sample holder.
Faraday cage with mesh lid: The Faraday cage with mesh lid consists of 3 parts: an open container, a mesh lid and a cap for securing the mesh lid to the open container. The open container is made of a conductive metal (e.g., steel, brass, aluminium etc) and sized to contain the plate or dish to be coated. The cap is also made from a conductive metal, conforms to the shape of the open container and is used to electrically connect the stainless-steel mesh lid to the open container (i.e., mesh lid is sandwiched between the cap and the open container). This forms a Faraday cage around the plate or dish. Three sizes of mesh, 4 mesh, 10 mesh and 50 mesh were investigated for effect of plasma treatment using the same sample holder and other parameters. The configuration and sizes of a Faraday cage that is suitable for holing a plate are shown in FIG. 7.

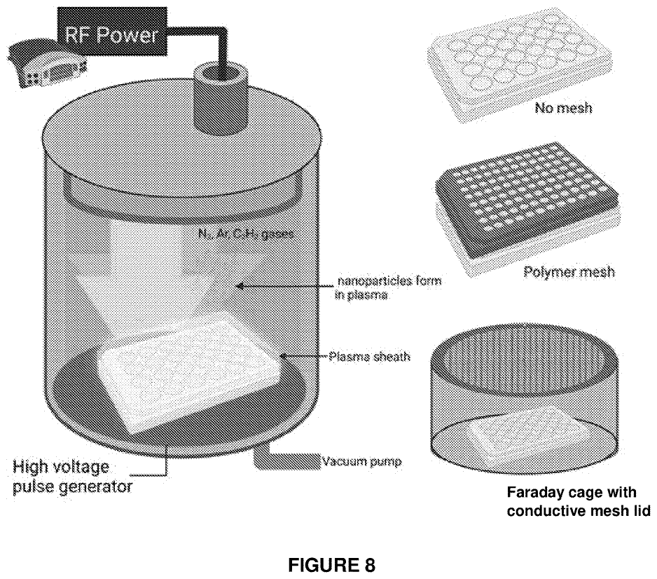

The plasma treatment was performed using a custom-made three-chamber system (FIG. 8). A culture plate (Corning Costar, 24 tissue culture plates) was positioned on the sample holder, either on a metal plate or in Faraday cage with mesh lid. The system was evacuated until a base pressure of 5×10⁵ Torr was reached. The plasma treatment consisted of two steps: surface activation with argon plasma and plasma deposition. Both steps utilized plasma generated by an RF power supply, with settings of 75 W for surface activation and 50 W for deposition. A pulsed negative bias of 500 V, at 3 kHz, and with duration of 20 μs was applied to the sample holder. During the surface activation step, the pressure of argon was regulated within the range of 70-80 mTorr. For the deposition step, a gas mixture comprising acetylene, nitrogen, and argon was maintained at 110 mTorr, with flow rates of 1, 3, and 10 sccm, respectively. Surface activation was performed for durations of 2, 5, and 10 minutes, while the deposition step had a fixed duration of 10 minutes.

The measurement of PAC thickness and the quantification of nanoparticles were performed using silicon wafers and glass coverslips placed inside the wells. The PAC thickness was determined using spectroscopic ellipsometry (J.A. Woollam M2000), while the number of nanoparticles was counted using an Olympus BMX-10 optical microscope. Since the nanoscale size of plasma particles and contaminating dust makes them difficult to identify with an optical microscope, we conducted further verification using a flow cytometer and a scanning electron microscope (SEM). SEM images were captured on silicon wafers using a Zeiss Sigma HD SEM at magnifications of 1000× and 5000×. To reduce surface charging and enable imaging, the samples were coated with a conductive gold layer with a thickness of 5 nm. In the SEM imaging process, an Inlens detector was employed with a working distance ranging from 3-5 mm. The field emission gun was set at 5 kV to facilitate the imaging process.

The number of nanoparticles was reduced when a mesh was used during the plasma treatment. The use of polymer mesh slightly reduced the particles. Conductive meshes are more efficient to prevent the particles compared to polymer mesh, especially when the mesh spacing size gets smaller. The Faraday cage with 50 mesh lid shows particle counts comparable to an untreated control plate (Table 1).

TABLE 4
Number of particles counted on each image on coverslips located on
different wells from each condition of plasma treatment. The results
are the average of 3 images taken on each glass coverslip.

| | | Particles count | | | | |
|---|---|---|---|---|---|---|
| | Well location | B1 | C3 | C4 | B6 | average |
| Untreated plate (positive control) | | 5.8 | 6.8 | 4.2 | 5.6 | 5.6 |
| Faraday cage | 50 mesh | 10.7 | 1.3 | 4.3 | 3.7 | 5 |
| | 10 mesh | 13.5 | 97.6 | 759.8 | 14.2 | 221.3 |
| | 4 mesh | 73.5 | 486 | 411.4 | 99.3 | 267.6 |
| Conductive mesh | 50 mesh | 56 | 136 | 61 | 25 | 69.5 |
| | 10 mesh | 158.5 | 265 | 346.4 | 153.3 | 230.8 |
| | 4 mesh | 8251 | 263 | 598 | 115 | 430.5 |
| Polymer mesh | 3 mm spacing | 149 | 779.8 | 5599 | lost | 2175.9 |
| | 4 mm spacing | 334 | 1338 | 1401 | 232 | 826.3 |
| | 5 mm spacing | 72.5 | 290.4 | 322.6 | 32.4 | 179.5 |

Flow cytometer Cytek Aurora (USA) was used to count the number of particles in solution. Wells of plasma treated 24-well plates, plasma treated with and without meshes, had 200 ml milliQ water added with to remove nanoparticles using 4 methods with increasing levels of shear force, as described below.
Still: gently add water to remove the particles
Pipette: use a pipette to withdraw and release water several times to remove the particles
Rocker: water was added to the samples and left on a rocker for 5 min to remove the particles
Sonication: high power sonication was applied for 5 min to remove the particles

Solutions with nanoparticles were then collected and analysed using flow cytometry (Cytek Aurora, USA). For each sample, a stopping volume of 15 μl was set and the number of nanoparticles detected were noted by FlowJo.

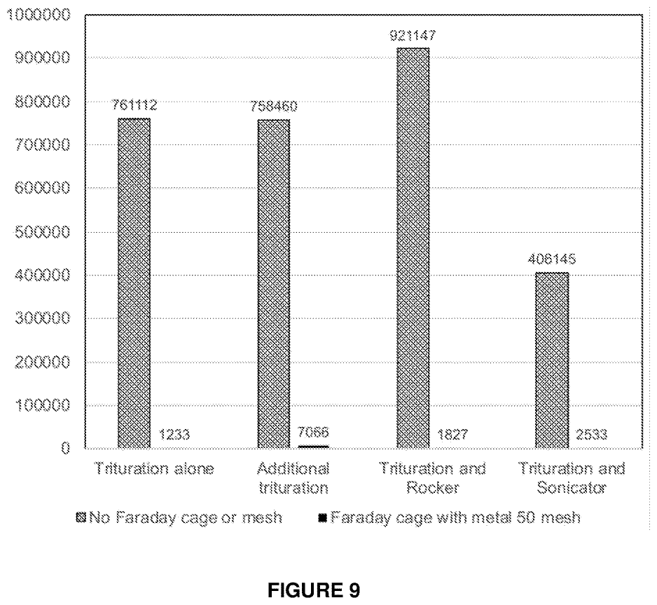

FIG. 9 compares the number of nanoparticles detected in 15 μl liquid using a flow cytometer, showing a significantly drop of particle count when plasma treatment was conducted with a Faraday cage with 50 mesh lid compared to conventional treatment without a mesh. The high density of nanoparticles in the liquid samples removed from no mesh treated plates indicates that these nanoparticles were loosely bound to the PAC and can easily be removed with low shear force such as the still method.

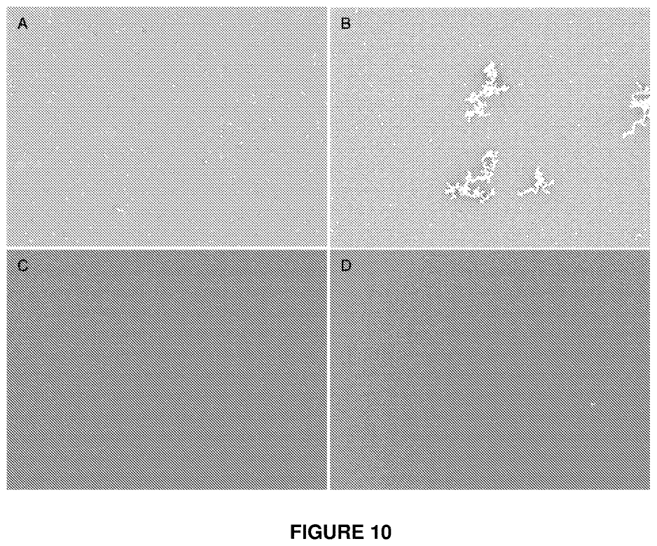

Nanoparticles forming on the coating were visualized from SEM images (FIG. 10). They are present as single particles or clumps of particles on the coating obtained from no mesh plasma treatment. In contrast, when using a Faraday cage with 50 mesh lid, very few nanoparticles can be found on the clear and smooth coating.

The coating thickness was determined by placing silicon wafers inside the wells and using spectroscopic ellipsometry to measure the PAC deposited on the surface of the silicon. FIG. 11 compares the PAC thickness when silicon wafers were positioned at various locations across the plate. The coating thickness was generally thicker using stainless steel meshes compared to polymer meshes, especially as the mesh size decreased. It is hypothesised that the polymer mesh cannot conduct away the charges that land on it so a positive charge builds up over time that reduces the electric field created by the negative bias and therefore the force drawing ions from the plasma to create the coating is also reduced. This is less of an issue for stainless steel meshes as the charges are conducted away.

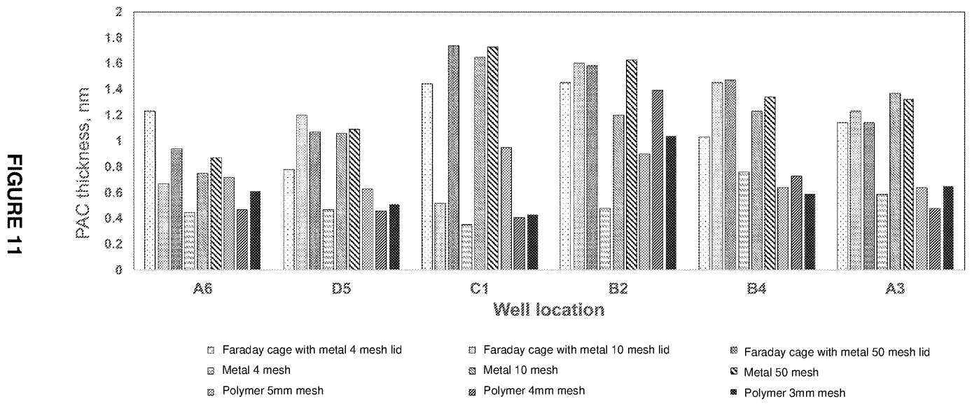

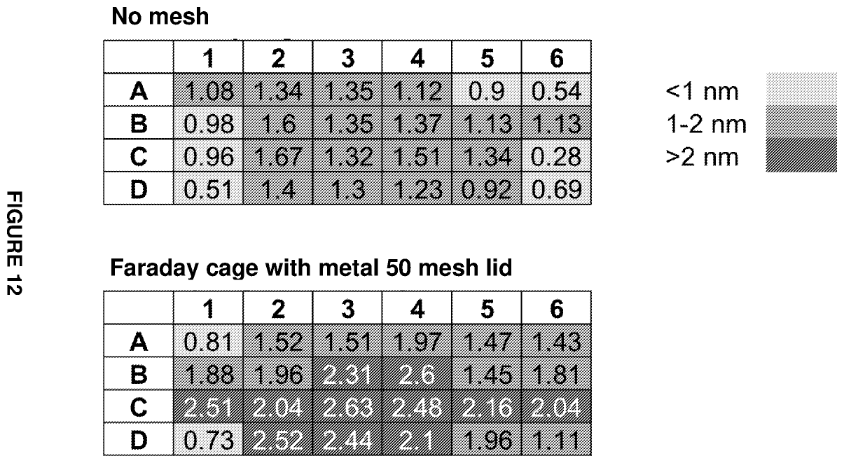

FIG. 12 provides a comprehensive comparison of the PAC thickness between treatment without a mesh and treatment with a Faraday cage with 50 mesh lid. Without using a mesh, the PAC thickness was measured to be 1.13±0.35 nm. However, when a mesh was used, the PAC thickness increased to 1.89±0.54 nm.

### Example 10—Comparison of Cell Culture in Plasma Treated Wells

The effect of plasma treatment on the differentiation of induced pluripotent stem cells (iPSCs) into cardiomyocytes (CM) was examined on 24 well plates that were untreated (UT), plasma treated without mesh (−Mesh) and plasma treated with a Faraday cage with 50 mesh lid (+Mesh), and then coated with Laminin 521 (LN521).

Briefly, Laminin-521 (LN521) was diluted in PBS (containing Ca²⁺ and Mg²⁺) to a concentration of 10 μg/mL and 200 μL added to each well of UT, −Mesh and +Mesh treated wells. After overnight incubation at 4 °C the wells were washed. Human induced pluripotent stem cells (iPSCs) (Gibco Episomal iPSC Line: A18945) that had been cultured on Matrigel in a 6-well plate (Corning Costar) in mTeSR Plus Basal Medium with Plus 5× Supplement added (STEMCELL, #100-0276) were passaged and plated into the wells at a density of approximately 3×10⁵ cells/well. The day after seeding (day −1), fresh mTeSR Plus Basal Medium with Plus 5× Supplement was added to dilute out ROCK inhibitor. The following day (day 0). CM differentiation media A (STEMCELL, #05010) was added for two days. Differentiation media B (STEMCELL, #05010) was added on day 2, followed by differentiation media C (STEMCELL, #05010) on days 4 and 6. Finally, the differentiation media was replaced with Cardiomyocyte Maintenance Basal Medium (STEMCELL, #05010) on day 8 and was replenished continuously every 2 days.

The progression of iPSCs into beating cardiomyocytes (CMs) was measured using a Nikon Microscope with a 4× objective. The quantity of beating colonies as a percentage of the visible area in the eyepiece was recorded in 9 different places and averaged to approximate the relative percentage of the total well which contained beating colonies. Each sample was performed in triplicates, giving n=3 data points to plot on GraphPad (GraphPad Software, version 9.5.1, San Diego, California).

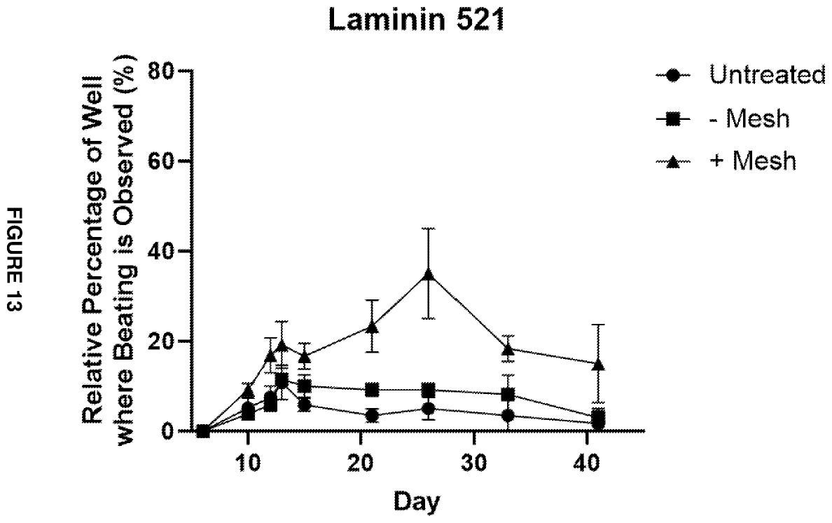

FIG. 13 shows that +Mesh provides a stable surface on which iPSCs can grow and differentiate into CMs. +Mesh is superior to −Mesh, which is in turn superior to UT. It is hypothesised that this is due to the increased number of nanoparticles in the UT and −Mesh treated wells to which the laminin binds and are washed away during media exchange—exposing areas without laminin coating. In contrast +Mesh wells have a even PAC coating to which the laminin covalently binds and fewer nanoparticles.

1. A method for coating a plastic surface, the method comprising:
(a) treating the plastic surface with ions under a pulsed bias; and
(b) depositing plasma on the plastic surface by plasma enhanced chemical vapour deposition (PECVD) of a gas mixture comprising acetylene and argon under a pulsed bias,
wherein a conductive mesh is positioned above the plastic surface such that the plasma passes through the mesh before being deposited on the plastic surface, and wherein the method produces a plasma-activated coating (PAC) on the plastic surface that covalently binds a biomolecule.

2. The method of claim 1, wherein ions are argon ions.

3. The method of claim 1, wherein the gas mixture further comprises nitrogen.

4. The method of claim 1, wherein the atmospheric percentage of nitrogen in the gas mixture is less than about 35%

5. The method of claim 1, wherein the atmospheric percentage of nitrogen in the gas mixture is about 21%.

6. The method of claim 1, wherein the treatment with ions is conducted for about 2 minutes at RF power of about 75 W and negative pulsed bias voltage of about 500 V.

7. The method of claim 1, wherein the plasma deposition is conducted for about 10 to about 30 minutes with a plasma discharge of about 50 W and a negative pulsed bias voltage of about 500 V.

8. The method of claim 1, wherein the negative pulsed bias is applied with a frequency of about 3 kHz and pulse duration of about 20 μs.

9. The method of claim 1, wherein the mesh is formed from a conductive metal.

10. The method of claim 1, wherein the mesh forms the lid of a Faraday cage enclosing the plastic surface.

11. The method of claim 9, wherein the conductive metal is stainless steel.

12. The method of claim 1, wherein the plastic surface has non-flat geometry.

13. The method of claim 1, wherein the plastic surface is a microplate well or a Petri dish.

14. The method of claim 1, wherein the plastic is polystyrene.

15. The method of claim 1, wherein the biomolecule is a nucleic acid or a protein.

16. The method of claim 1, wherein the PAC has a property selected from any one or more of the following:
a surface nitrogen concentration of about 4 to about 14%;
a relative density of radicals of about 0.016 to about 0.021;
a mean absorption at 450 nm of about 0.052 to about 0.056 a.u.;
a thickness of about 1 nm to about 10 nm;
hybridises DNA at a concentration of about 2×10¹¹ to about 5×10¹¹ molecules/cm²; and
immobilises streptavidin at a concentration of about 2×10¹¹ to about 9×10¹¹ molecules/cm².

17. A plastic surface coated with a plasma-activated coating (PAC), wherein the PAC covalently binds a biomolecule and has a property selected from any one or more of the following:
a surface nitrogen concentration of about 4 to about 14%;
a relative density of radicals of about 0.016 to about 0.021;
a mean absorption at 450 nm of about 0.052 to about 0.056 a.u.;
a thickness of about 1 nm to about 10 nm;
hybridises DNA at a concentration of about 2×10¹¹ to about 5×10¹¹ molecules/cm²; and
immobilises streptavidin at a concentration of about 2×10¹¹ to about 9×10¹¹ molecules/cm².
